(19) United States
(12) Patent Application Publication
(10) Pub. No.: US 2018/0030389 A1
(43) Pub. Date: Feb. 1, 2018

(54) Multi-Purpose Cleaner System

(71) Applicants: Amit Garyali, Oakville (CA); Darshna Ramji, Oakville (CA)

(72) Inventors: Amit Garyali, Oakville (CA); Darshna Ramji, Oakville (CA)

(21) Appl. No.: 15/224,477

(22) Filed: Jul. 29, 2016

(51) Int. Cl. C11D 9/02; C11D 9/42; A61K 8/92; A61K 8/36; A01N 25/02; A61K 8/22; A61Q 19/10; A01N 59/00; A01N 37/02; A01N 31/02; C11D 9/26; A61K 8/34

(52) U.S. Cl. CPC C11D 9/02; C11D 9/26; C11D 9/42; A61K 8/92; A61K 8/36; A61K 8/34; A61K 8/22; A61Q 19/10; A01N 59/00; A01N 37/02; A01N 31/02; A01N 25/02

(57) ABSTRACT

A multi-purpose cleaner system for providing a composition for cleaning at least one surface. The multi-purpose cleaner composition is all natural and cleans a variety of surfaces like flooring, glass, counter tops, porcelain, grout, and many more. At least one essential oil is included in the multi-purpose cleaner composition to provide aromatherapy benefits. The multi-purpose cleaner system is intended for continuous use in order to deliver healthier and aesthetically pleasing surfaces.

### BACKGROUND OF THE INVENTION

The following includes information that may be useful in understanding the present invention(s). It is not an admission that any of the information provided herein is prior art, or material, to the presently described or claimed inventions, or that any publication or document that is specifically or implicitly referenced is prior art.

### 1. FIELD OF THE INVENTION

The present invention relates generally to the field of cleaning compositions and more specifically relates to a multi-purpose cleaner system.

### 2. DESCRIPTION OF RELATED ART

A variety of cleaning formulations are available for household use on hard surfaces such as countertops, for use on textiles on upholstery and carpets, for example. Still other cleaning formulations are used on hands to remove dirt and greases, etc. Several considerations in formulating such compositions include the cleaning ability to dissolve stains and dirt as well as the ability to remove them. For example, in surface cleaning formulations for carpeting, some conventional cleaning compositions interact with the dirt or stain, and the residue can be later removed.

Because of the significant dangers posed by synthetic cleaners and the ever increasing desire for renewable resources, there has been an increased demand for entirely natural cleaning compositions. However, past natural based cleaning compositions have suffered from shortcomings. Many such compositions have used all natural ingredients but failed to perform at the level of synthetically composed cleaners, forcing consumers to choose between their health and effective cleaning. Secondly, many other compositions have achieved similar performance to that of the synthetically composed cleaners but only use several natural ingredients in combination with several synthetic surfactants and solvents. Therefore, a suitable multi-purpose, all natural cleaning solution is desired.

Several attempts have been made to solve the above-mentioned problems such as those found in U.S. Pub. No. 2016/0010037 to Peter Nelson Wainman, et al. and U.S. Pat. No. 7,465,697 to Samuel DeAth; U.S. Pat. No. 8,206,761 to Dzovig M. Kutumian; U.S. Pat. No. 7,396,808 to Ryan K. Hood, et al.; U.S. Pat. No. 7,741,261 to Ivania Guerrero; U.S. Pat. No. 6,432,395 to Michael R. Rochon, et al. This art is representative of cleaning compositions. However, none of the above inventions and patents, taken either singly or in combination, is seen to describe the invention as claimed.

### BRIEF SUMMARY OF THE INVENTION

In view of the foregoing disadvantages inherent in the known cleaning composition art, the present invention provides a novel multi-purpose cleaner system. The general purpose of the present invention, which will be described subsequently in greater detail is to provide a means for cleaning at least one surface.

A multi-purpose cleaner system is disclosed herein, in a preferred embodiment, comprising a multi-purpose cleaner composition comprising distilled vinegar, isopropyl alcohol, at least one essential oil, hydrogen peroxide 10%, castile liquid soap, and boiled tap water. The multi-purpose cleaner system comprises the multi-purpose cleaner composition. The multi-purpose cleaner composition comprises in functional combination the distilled vinegar, the isopropyl alcohol, the at least one essential oil, the hydrogen peroxide 10%, the castile liquid soap, and the boiled tap water thereby creating an admixture.

The at least one essential oil may provide aromatherapy benefits. The at least one essential oil may comprise eucalyptus scent. The multi-purpose cleaner composition is suitably mixed to provide a means for cleaning at least one surface. The at least one surface comprises flooring or the like. The at least one surface may be selected from the group consisting of the glass, the flooring, the porcelain, the stainless steel, the plastic, and the counter tops. The multi-purpose cleaner composition is preferably anti-microbial and anti-fungal. The multi-purpose cleaner composition is all-natural so does not leave an environmental footprint.

According to another embodiment, a method for using multi-purpose cleaner system is also disclosed herein. The method for providing a multi-purpose cleaner composition includes the steps of: applying an admixture solution comprising the multi-purpose cleaner composition to at least one surface, cleaning at least one surface, and storing multi-purpose cleaner composition.

Methods for using multi-purpose cleaner system varies upon surface. Surfaces, such as flooring, use dry mopping method. The user is to always shake or mix multi-purpose cleaner system before use. The dry mopping method comprises applying the multi-purpose cleaner composition to a surface(s) and wiping the composition with quick even strokes; wipe along the wood grain patterns for hard wood, laminate, vinyl, etc. For best results, users should not walk over or use area being cleaned for at least 20 minutes. Once waiting period has been completed, surface(s) cleaned may remain clear and shiny for approximately two weeks.

For all other surfaces, a cotton cloth, magic eraser, towel, or paper towel may be utilized. Methods of using cotton cloth, towel, magic eraser, or paper towel varies upon surface(s). Users may experiment and utilize whichever tool achieves best results. For porous surfaces (natural wood furniture, hardwood & natural stones etc.) allow multi-purpose cleaner composition to settle on surface for a maximum of 30 to 45 seconds; for non-porous surfaces (grout, tiles & ceramics etc.) allow composition to settle for a maximum of five minutes before wiping dry. Preferably, dry food particles may be wiped away before using multi-purpose cleaner composition. Paper towels or cotton towels may be desirable for stainless steel. Continuous use of multi-purpose cleaner system provides healthier and shinier surfaces. Surface(s) that may not be used with the multi-purpose cleaner system include: fabrics (i.e. cotton, rayon, nylon, linen, micro fiber, and carpet) and walls.

The present invention holds significant improvements and serves as a multi-purpose cleaner system. Preferably, a cleaning composition should provide a means for cleaning at least one surface and, yet would operate reliably and be manufactured at a modest expense. Thus, a need exists for a reliable multi-purpose cleaner system to avoid the above-mentioned problems.

For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and method(s) of use for the present disclosure, multi-purpose cleaner system, constructed and operative according to the teachings of the present disclosure.

FIG. 1 shows a perspective view illustrating a multi-purpose cleaner system during an ‘in-use’ condition according to an embodiment of the disclosure.

FIG. 2 is a perspective view illustrating the multi-purpose cleaner system during an ‘in-use’ condition according to an embodiment of the disclosure.

FIG. 3 is a perspective view illustrating the multi-purpose cleaner system during an ‘in-use’ condition according to an embodiment of the disclosure.

FIG. 4 is a perspective view illustrating at least one surface according to an embodiment of the present disclosure.

FIG. 5 is a flow diagram illustrating a method of use for the multi-purpose cleaner system according to an embodiment of the present disclosure.

The various embodiments of the present invention will hereinafter be described in conjunction with the appended drawings, wherein like designations denote like elements.

### DETAILED DESCRIPTION

As discussed above, embodiments of the present disclosure relate to a cleaning composition and more particularly to a multi-purpose cleaner system as used to improve the effective means for cleaning at least one surface.

Generally speaking, multi-purpose cleaner system incorporates all natural ingredients and essential oils. Essential oils may be pure, tested, ethically sourced, or vegan. Other essential oils that may be included in the composition are bergamot, cedar wood, clementine, grapefruit pink, lemon, and orange sweet. Essential oils may be combined into multi-purpose cleaner system or included individually. When used individually or as a combination, essential oils comprise 11.9% of the multi-purpose cleaner composition. Essential oils found in the multi-purpose cleaner composition provide aromatherapy treatment which includes but is not limited to calming, de-stressing, mood lifting, energizing, improved breathing, and various of other benefits. To receive all benefits from multi-purpose cleaner system, multi-purpose cleaner composition must be in a state of agitation during use so that all ingredients may be properly mixed.

A variety of surfaces used with multi-purpose cleaner system may include: flooring (i.e., hardwood, laminate, vinyl, marble, ceramic, stone, granite, etc.), grout & leather. The multi-purpose cleaner system may also be used to remove glue, permanent marker, colored markers, paint and numerous of other surfaces.

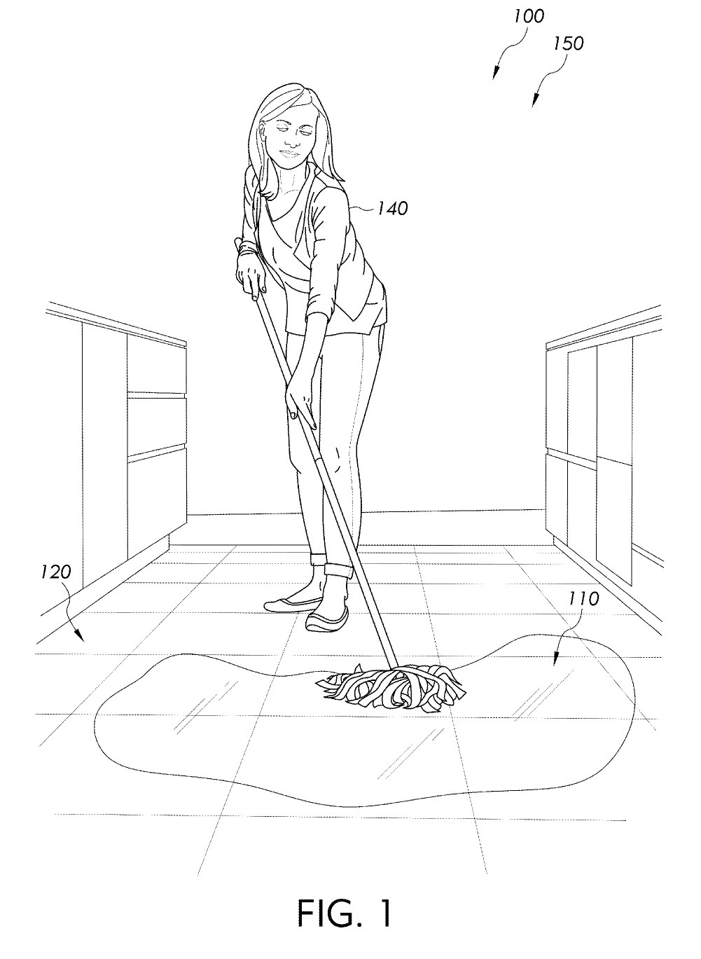

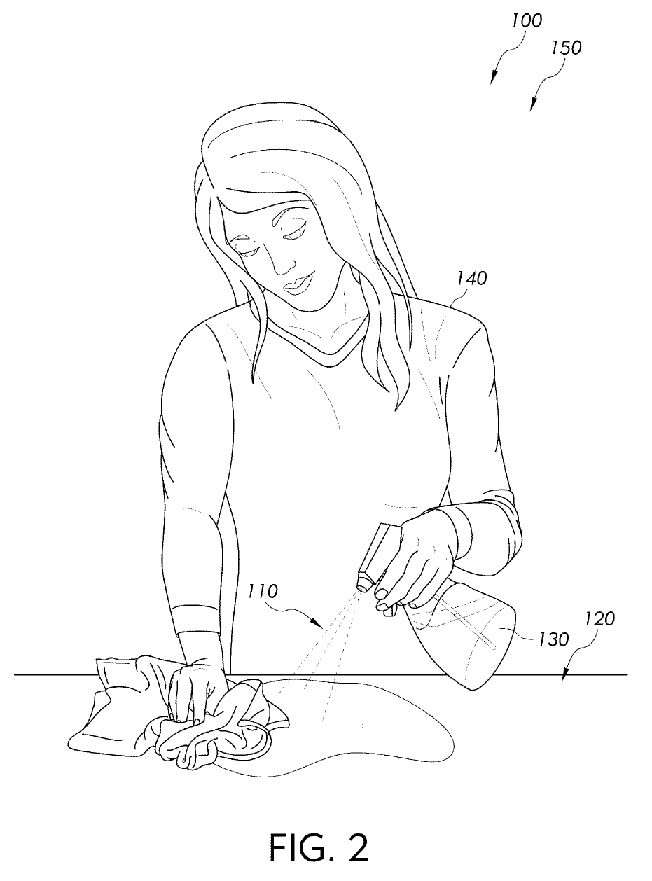

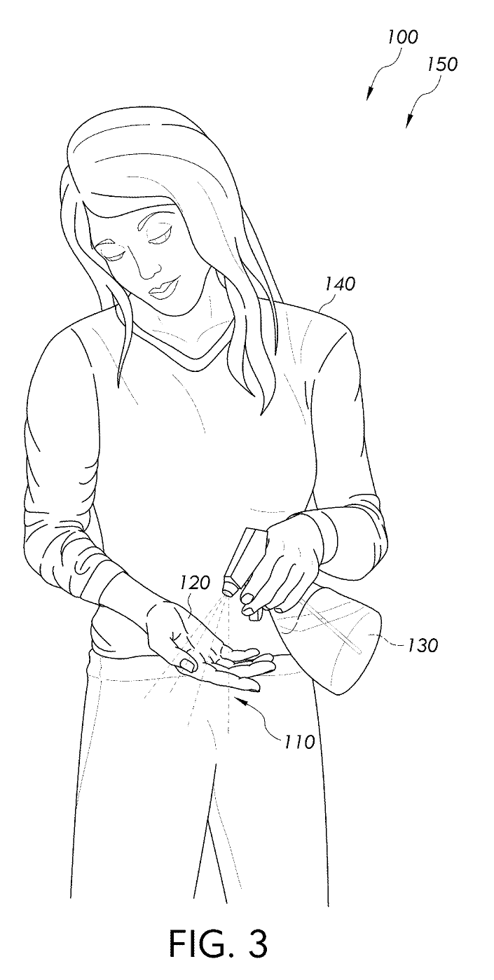

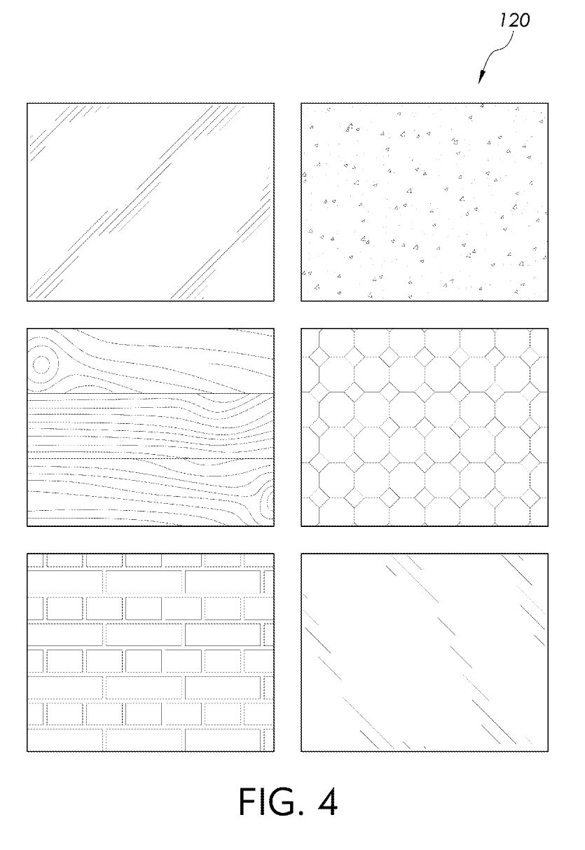

Referring now more specifically to the drawings by numerals of reference there is shown in FIGS. 1-4, various views of multi-purpose cleaner system 100 comprising: multi-purpose cleaner composition 110 comprising distilled vinegar, isopropyl alcohol, at least one essential oil, hydrogen peroxide 10%, castile liquid soap, and boiled tap water. Multi-purpose cleaner system 100 comprises multi-purpose cleaner composition 110. Multi-purpose cleaner composition 110 comprises in functional combination the distilled vinegar, the isopropyl alcohol, the at least one essential oil, the hydrogen peroxide 10%, the castile liquid soap, and the boiled tap water thereby creating an admixture in certain embodiments.

Multi-purpose cleaner composition 110 is suitably mixed to provide a means for cleaning at least one surface 120. At least one surface 120 may comprise glass, flooring, porcelain, plastic, stainless steel, counter tops, or hands Multi-purpose cleaner composition 110 is anti-microbial. Multi-purpose cleaner composition 110 may be anti-fungal. At least one surface 120 does not comprise walls. At least one surface 120 does not comprise fabric. At least one essential oil 130 provides aromatherapy benefits. At least one essential oil 130 may comprise citrus scent. At least one essential oil 130 may comprise eucalyptus scent. Multi-purpose cleaner composition 110 is all-natural.

Upon reading this specification, it should be appreciated that, under appropriate circumstances, considering such issues as user preferences, marketing preferences, cost, available materials, technological advances, etc.

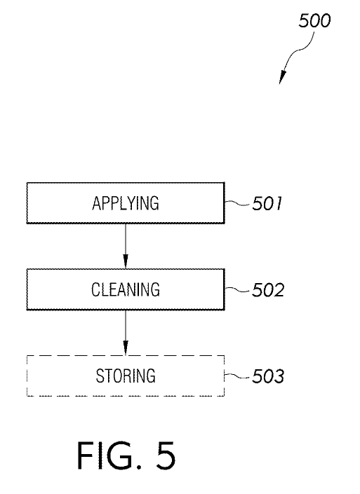

Referring now to FIG. 5 showing a flow diagram 550 illustrating method of use 500 for multi-purpose cleaner system 100 according to an embodiment of the present invention of FIGS. 1-4. As shown, method of use 500 may comprise the steps of: step one 501, applying an admixture solution comprising multi-purpose cleaner composition 110 to at least one surface 120; step two 502, cleaning at least one surface 120; step three 503, storing multi-purpose cleaner composition 110. Other methods may be used such as those described previously herein.

It should be noted that step three 503 is an optional step and may not be implemented in all cases. Optional steps of method of use 500 are illustrated using dotted lines in FIG. 5 so as to distinguish them from the other steps of method of use 500.

It should be noted that the steps described in the method of use can be carried out in many different orders according to user preference. The use of “step of” should not be interpreted as “step for”, in the claims herein and is not intended to invoke the provisions of 35 U.S.C. §112 (f). Upon reading this specification, it should be appreciated that, under appropriate circumstances, considering such issues as design preference, user preferences, marketing preferences, cost, structural requirements, available materials, technological advances, etc., other methods of use arrangements such as, for example, different orders within above-mentioned list, elimination or addition of certain steps, including or excluding certain maintenance steps, etc., may be sufficient.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is new and desired to be protected by Letters Patent is set forth in the appended claims:

1. A multi-purpose cleaner system comprising:
a multi-purpose cleaner composition comprising;
distilled vinegar,
isopropyl alcohol,
at least one essential oil,
hydrogen peroxide 10%,
castile liquid soap, and
boiled tap water;
wherein said multi-purpose cleaner system comprises said multi-purpose cleaner composition;
wherein said multi-purpose cleaner composition comprises in functional combination said distilled vinegar, said isopropyl alcohol, said at least one essential oil, said hydrogen peroxide 10%, said castile liquid soap, and said boiled tap water thereby creating an admixture; and
wherein said multi-purpose cleaner composition is suitably mixed to provide a means for cleaning at least one surface.

2. The multi-purpose cleaner system of claim 1 wherein said at least one surface comprises glass.

3. The multi-purpose cleaner system of claim 1 wherein said at least one surface comprises flooring.

4. The multi-purpose cleaner system of claim 1 wherein said at least one surface comprises porcelain.

5. The multi-purpose cleaner system of claim 1 wherein said at least one surface comprises plastic.

6. The multi-purpose cleaner system of claim 1 wherein said at least one surface comprises stainless steel.

7. The multi-purpose cleaner system of claim 1 wherein said at least one surface comprises counter tops.

8. The multi-purpose cleaner system of claim 1 wherein said at least one surface comprises hands.

9. The multi-purpose cleaner system of claim 1 wherein said multi-purpose cleaner composition is anti-microbial.

10. The multi-purpose cleaner system of claim 1 wherein said multi-purpose cleaner composition is anti-fungal.

11. The multi-purpose cleaner system of claim 1 wherein said at least one surface does not comprise walls.

12. The multi-purpose cleaner system of claim 1 wherein said at least one surface does not comprise fabric.

13. The multi-purpose cleaner system of claim 1 wherein said at least one essential oil provides aromatherapy benefits.

14. The multi-purpose cleaner system of claim 1 wherein said at least one essential oil comprises citrus scent.

15. The multi-purpose cleaner system of claim 1 wherein said at least one essential oil comprises eucalyptus scent.

16. The multi-purpose cleaner system of claim 1 wherein said multi-purpose cleaner composition is all-natural.

17. A multi-purpose cleaner system comprising:
a multi-purpose cleaner composition comprising;
distilled vinegar,
isopropyl alcohol,
at least one essential oil,
hydrogen peroxide 10%,
castile liquid soap, and
boiled tap water;
wherein said multi-purpose cleaner system comprises said multi-purpose cleaner composition;
wherein said multi-purpose cleaner composition comprises in functional combination said distilled vinegar, said isopropyl alcohol, said at least one essential oil, said hydrogen peroxide 10%, said castile liquid soap, and said boiled tap water thereby creating an admixture;
wherein said at least one essential oil aromatherapy benefits;
wherein said at least one essential oil comprises eucalyptus scent;
wherein said multi-purpose cleaner composition is suitably mixed to provide a means for cleaning at least one surface;
wherein said at least one surface comprises flooring;
wherein said multi-purpose cleaner composition is anti-microbial;
wherein said multi-purpose cleaner composition is anti-fungal; and
wherein said multi-purpose cleaner composition is all-natural.

18. The multi-purpose cleaner system of claim 17 wherein said at least one surface is selected from the group consisting of said glass, said flooring, said porcelain, said stainless steel, said plastic, and said counter tops.

19. A method of using a multi-purpose cleaner system comprising the steps of:
applying an admixture solution comprising said multi-purpose cleaner composition to at least one surface; and
cleaning said at least one surface;

20. The method of claim 19 further comprising the step of:
storing said multi-purpose cleaner composition
